(19) United States
(12) Patent Application Publication
(10) Pub. No.: US 2012/0042416 A1
(43) Pub. Date: Feb. 16, 2012

(54) Antibody fusion-mediated plant resistance against Oomycota

(75) Inventors: Sylvia Schleker, Duren (DE); Dieter Peschen, Geilenkirchen (DE); Rainer Fischer, Aachen (DE); Stefan Schillberg, Aachen (DE)

(73) Assignee: Fraunhofer-Gesellschaft zur Forderung der angewandten e.V., Munchen (DE)

(21) Appl. No.: 13/264,224

(22) PCT Filed: Aug. 5, 2010
(86) PCT No.: PCT/EP10/61427

§ 371 Date: Oct. 13, 2011

(30) Foreign Application Priority Data
Aug. 24, 2009 (EP) 09168451.4

(51) Int. Cl. A01H 5/00; C12N 15/62; C12P 21/02; C12N 15/82; A01H 1/06; C12N 5/10; A01N 37/18; A01P 3/00; A01N 43/90; C40B 40/10; G01N 33/566; C12Q 1/04; C12N 1/21; C12N 1/19; C12P 21/00; A01N 43/50; C12Q 1/68; C07K 19/00

(52) U.S. Cl. 800/279; 530/387.3; 530/391.3; 536/23.4; 435/320.1; 435/69.6; 435/468; 800/301; 435/418; 424/183.1; 514/44 R; 506/18; 435/7.92; 435/34; 435/252.3; 435/254.2; 435/348; 435/419; 435/6.12

(57) ABSTRACT

The present invention relates to fusion proteins comprising anti-Oomycotic proteins or peptides linked to an antibody or fragment thereof specifically recognising an epitope of an Oomycota. The invention is also directed to polynucleotides coding for the fusion proteins. The embodiments of the present invention are particularly useful for the protection of plants against Oomycota. The invention therefore comprises transgenic plants expressing the fusion proteins of the present invention.

The present invention relates to fusion proteins comprising anti-Oomycotic proteins or peptides linked to an antibody or fragment thereof specifically recognising an epitope of an Oomycota. The invention is also directed to polynucleotides coding for the fusion proteins. The embodiments of the present invention are particularly useful for the protection of plants against Oomycota. The invention therefore comprises transgenic plants expressing the fusion proteins of the present invention.

Plant disease constitutes a major and ongoing threat to human food stocks and animal feed. Most crop plants are regularly exposed to one or more pathogen(s) that can cause incredible damage resulting in substantial economical losses every year. Attack by pathogens, such as viruses, bacteria, fungi, Oomycota, nematodes and insects is a severe economic problem, which impacts all economically important crops, for example, potato, tomato, vegetables, trees as oak and eucalyptus, fruit trees, cut flowers and ornamental plants. Current protective measures rely heavily on chemical control measures for pathogens, which have undesirable environmental consequences. Natural based resistance against Oomycota often not exists.

A more effective approach to protecting plants from pathogen attack is to create plants that are endogenously resistant to Oomycota.

However, plant breeders have limited sources of resistance genes against plant diseases. This can now be achieved using genetic engineering techniques, by providing the plant with genetic information required for affecting the pathogens and for being resistant to the disease caused by the pathogen. For example, in the case of an Oomycota pathogen, the host plant is resistant if it has the ability to inhibit or retard the growth of an Oomycota, the symptoms of Oomycota infection or the life cycle of the Oomycota, including its spreading.

WO-A-00/23593 describes the general idea of providing plants with pathogen-resistance by expressing fusions of a binding domain directed against a plant pathogen with a domain which is toxic for the respective plant pathogen. According to WO-A-03/086475 a plant pathogen is a virus or virus-like organism, bacterium, mycoplasma, fungus, insect or nematode. However, only examples of fusions against plant viruses were demonstrated.

WO-A-03/089475 discloses fusion constructs of an antifungal protein or peptide (“AFP”) and an antibody or antibody fragment directed against an Ascomycota together with a cellular targeting sequence.

There is still a need for a safe and reliable protection of plants against specific pathogens. In particular, to date no such effective protection of plants against Oomycota exists. Oomycota are very distinct plant pathogens, in particular in comparison to fungal pathogens such as Ascomycota. It is therefore the technical problem underlying the present invention to provide a safe and reliable means for protecting plants against oomycotic pathogens or related pathogens.

Oomycota are fungi like pathogens but are related to organisms such as brown algae and diatoms, making up a group called the heterokonts which does not belong to the kingdom of fungi. Compared to Ascomycota and Basidiomycota Oomycota show a number of differences. They evolved separately, for instance, their cell walls are composed of cellulose rather than chitin and generally do not have septations. Also, in the vegetative state they have diploid nuclei, whereas fungi have haploid nuclei. They typically produce asexual spores called zoospores, which capitalize on surface water for movement. The sexual spores, called oospores, that are translucent double-walled spherical structures are used to survive adverse environmental conditions. Compared to Oomycota Ascomycota are marked by a characteristic structure, the ascus, which distinguishes these fungi from all others. An ascus is a tube-shaped vessel, a meiosporangium, which contains the sexual spores produced by meiosis. Typically all Ascomycota are haploid, so their nuclei only contain one set of chromosomes.

The solution to the above technical problem is provided by the embodiments of the present invention as defined in the claims.

In particular, the present invention provides a fusion protein comprising at least one anti-Oomycotic protein or peptide (AOP) linked to an antibody or fragment thereof specifically recognising an epitope of an Oomycota. Thus, the fusion protein according to the present invention is an immune-pesticide having an affinity portion against Oomycota surface structure(s) and an anti-Oomycotic protein portion or an anti-Oomycota peptide portion.

Preferably, the AOP is selected from the group consisting of Cec, D4E1, GR7, Mag, and Metchnikowin (MTK). Specific sequences of anti-Oomycota peptides according to the invention may be selected from SEQ ID NO: 70 to SEQ ID NO: 75. Corresponding nucleotide sequences are disclosed in SEQ ID NO: 10 to SEQ ID NO: 14.

It is further preferred that the antibody or fragment thereof specifically recognises an epitope of Phytophthora ssp., preferably Phytophthora infestans and/or Phytophthora nicotianae, Phytophthora cactorum, Phytophthora capsici, Phytophthora cinnamoni.

According to preferred embodiments of the fusion construct of the invention, the antibody or fragment thereof is a full-length antibody, F(ab′)₂ fragment, Fab fragment, scFv, bi-specific scFv, tri-specific scFv, diabody, single domain antibody (dAb), minibody or molecular recognition unit (MRU).

It is further preferred that the antibody fragment is an scFv having a sequence selected from the group consisting of SEQ ID NO: 61 to SEQ ID NO: 69.

Generally, the fusion protein according to the invention further comprises non or at least one, preferably N-terminal and/or C-terminal, tag at least facilitating the detection and/or purification of the fusion protein. More preferred, especially in case the affinity portion of the fusion protein is an scFv species, the antibody fragment according to the invention comprises one or more N-terminal and/or C-terminal tag(s). Specific examples of such tags include, but are not limited to, c-myc, his₆, his₅, tag54, FLAG, HA, HSV-, T7, S, strep and E-tag.

The fusion protein according to the present invention preferably further contains a cellular targeting sequence such as a sequence for secretion or location of the fusion protein to cell compartments or organelles, preferably the apoplast, the vacuole, intra- and/or exterior membranes or the ER lumen. Thus, the antibody (=“Ab”), recombinant antibody (=“rAb”), rAb fragments or fusions can be targeted to the apoplast or to organelles and plant cell compartments or immobilized and membrane anchored by addition of targeting sequences and/or membrane anchors.

It is further preferred that the AOP and the antibody or fragment thereof are linked by a peptide linker.

Specific preferred examples of fusion proteins according to the present invention are selected from the sequences according to SEQ ID NO: 76 to SEQ ID NO: 120.

The present invention also relates to a polynucleotide comprising a sequence encoding the fusion protein as defined herein.

Preferred polynucleotides of the present invention have a sequence that is optimised for expression the encoded fusion protein and/or propagation of the polynucleotide in a host cell. Optimisation of the sequence of the polynucleotide of the present invention includes parameters such as codon usage, GC content, repeat sequences (direct repeat, reverse repeat, Dyad repeat), CpG dinucleotides content, cryptic splicing sites, recombination sites, premature PolyA sites, internal chi sites and ribosomal binding sites, negative CpG islands, RNA instability motif (ARE), mRNA secondary structure and stability, RNA stabilising elements, nuclear translocation supporting sequence elements, restriction sites that may interfere with cloning etc.

Further subject matter of the present invention relates to a vector comprising the inventive polynucleotide, preferably within an expression cassette. More preferably, the expression cassette is operatively linked to one or more regulatory sequence(s) allowing the expression of the fusion protein, preferably in plants, plant organs, plant tissues and/or plant cells. Regulatory sequences in this context include, but are not limited to, promoters, enhancers, cis-acting elements, trans-acting factors which are preferably all optimised for expression of the fusion protein in the respective host. Further sequence elements for improvement of expression of the fusion proteins of the invention include the presence of Kozak and Shine-Dalgarno sequences.

It is clear for the person skilled in the art that such regulatory sequences can be present in the vector of the present invention, but can be also present in the polynucleotide of the invention as such, i.e. independent of incorporation of the polynucleotide into a vector.

The present invention also provides a host cell comprising the above-defined polynucleotide and/or vector. Host cells according to the invention include bacteria such as commercially available strains for cloning and/or expression of the present constructs, yeasts, insect cells, and, most preferred, plant cells such as cells from Solanum ssp. or Nicotiana ssp.

Further subject matter of the present invention is a method for the production of the above-defined fusion protein comprising the steps of:
(a) culturing the host cells of the present invention in a culture medium under conditions allowing the expression of the fusion protein; and
(b) recovering the fusion protein from the medium and/or the host cells.

The present invention further provides a method for the production of a Oomycota-resistant plant, plant cell or plant tissue comprising the step of introducing the polynucleotide of the present invention into the genome of the plant, plant cell or plant tissue.

The present invention is also directed to a transgenic plant or plant tissue transformed with the polynucleotide of the invention, and to harvestable and propagation materials derived from such transgenic plants or tissues. Preferred transgenic plants according to the present belong e.g. to the genera Solanum tuberosum or Nicotiana ssp.

The present invention further provides the use of the polynucleotide and/or the vector for the protection of a plant against the action of Oomycota.

The present invention is also directed to a kit comprising the above-defined fusion protein and/or the polynucleotide and/or the vector together with means for the detection of said fusion protein, vector and/or polynucleotide.

According to the invention it is possible to, e.g. express AOP-scFv fusions in potato, which induces resistance against P. infestans. This system poses a large threat for crops vegetables, trees as oak and eucalyptus, fruit trees, cut flowers and ornamental plants, which can hitherto only be protected by using high priced pesticides which have major negative impacts on the environment.

The term “antibody” and “antibody fragment” is used to denote polypeptide chain(s) which exhibit a strong monovalent, bivalent or polyvalent binding to a given epitope or epitopes. The antibodies may be generated by any suitable technology, such as hybridoma technology, or ribosome display, or phage display, of natural naïve origin, or immunized origin, semi-synthetic or fully synthetic libraries or combinations thereof. The term “antibody” is also used to denote designer antibodies. These antibody polypeptides are encoded by an immunoglobulin gene or immunoglobulin genes, or fragments thereof which specifically bind the given epitope or epitopes. The recognized immunoglobulin genes include the κ and λ light chain genes, the μ, δ, γ, α and ε constant regions as well as all immunoglobulin variable regions from vertebrate, camelid, avian and pisces species. The term antibody, as used herein, includes in particular those antibodies synthesized or constructed de novo using recombinant DNA methodology, such as recombinant full-size antibodies, dimeric secretory IgA antibodies, multimeric IgM antibodies, F(ab′)₂-fragments, Fab-fragments, Fv-fragments, single chain Fv-fragments (scFvs), bispecific scFvs, diabodies, single domain antibodies (dAb), minibodies (Vaughan and Sollazzo, 2001) and molecular recognition units (MRUs). Antibody sequences may be derived from any vertebrate, camelid, avian or pisces species using recombinant DNA technology, or also by using synthetic, semi-synthetic and naÏve or immunocompetent phage and ribosome display libraries, gene shuffling libraries, molecular evolution, and fully synthetic designer antibodies. In this invention, the antibodies are generated against specific pathogen or host plant epitopes that are involved in the pathogen growth, reproduction or life cycle.

The term AOP (anti-Oomycotic peptide or polypeptide) refers to an activity that affects the reproduction or growth of at least an Oomycota and/or any stages of its life cycle. In the case of Oomycota pathogens, this includes germination of spores, adhesion to the plant surface, entry into the plant, formation of appressoria and haustoria, penetrating a plant cell tissue or spreading. Antibodies or recombinant proteins in themselves are also considered toxic when they affect the Oomycota by binding to it and or host proteins that are utilized by a pathogen during its growth, reproduction, life cycle or spreading.

Monoclonal antibodies (Köhler and Milstein (1975) Nature. 256:495-497) can be raised against almost any epitope or molecular structure of a pathogen or host protein using several techniques. The most common method is the hybridoma technique starting with immunocompetent B lymphocytes from the spleen or thymus which are obtained after immunization with native antigen, recombinant antigen, antigen fusion proteins, antigen domains or by in vitro or genetic immunization. In addition, recent advances in molecular biology techniques now permit the use of cloned recombinant antibody fragments and antibodies derived from mice and other organisms than the mouse. Suitable recombinant antibody fragment(s) include the complete recombinant full-size antibodies, dimeric secretory IgA antibodies, multimeric IgM antibodies, the F(ab′)₂ fragment, the Fab-fragment, the Fv-fragment, single chain antibody fragments (scFvs), single binding domains (dAbs), a bivalent scFv (diabody), minibody, and bispecific scFv antibodies where the antibody molecule recognises two different epitopes, which may be from the pathogen or the host or both the pathogen and the host, triabodies or other multispecific antibodies and any other part of the antibody such as, molecular recognition units (MRUs), which show binding to the target epitopes. Genes encoding these suitable recombinant antibody fragment(s) may be derived from vertebrates, camelids, avian or pisces species or are synthetic.

Also, single chain antibodies (scFvs) that have affinities for pathogen or host structures and proteins can be identified using phage display libraries or ribosome display libraries, gene shuffled libraries, which can be constructed from synthetic, semi-synthetic or naïve and immunocompetent sources. Phage display and suitable techniques can be used to specifically identify antibodies, or fragments thereof, with the desired binding properties. Using recombinant antibody technology it is possible to identify antibodies or fragments that are highly specific for a single pathogen, or which recognize a consensus epitope conserved between several pathogens, where the antibodies will have a broad specificity against pathogens. The durability and effect of antibody mediated resistance can be improved by i) recombinant antibody affinity maturation, ii) CDR randomization and selection, iii) stabilization by framework optimization of a selected pathogen specific antibody, iv) bi-specific antibody expression, v) the generation of antibody fusion proteins, or vi) the expression of antibodies in combinations with others that may potentiate their individual effects. For example, surface plasmon resonance as employed in the BIAcore system can be used to increase the efficiency of phage displayed antibodies selections, yielding a high increment of affinity from a single library of phage antibodies which bind to an epitope of a pathogen with desired on- and off-rates.

The recombinant antibodies can be identified and utilized according to methods that are familiar to anyone of ordinary skill in the art.

This invention describes antibodies or fragments thereof which recognize structures of Oomycota directly or indirectly leading to resistance or partial resistance when expressed as a chimeric fusion protein coupled to an anti-Oomycota activity and/or coexpression of several of these constructs.

Antibodies can be generated that recognize Oomycota-specific epitopes or host plant-specific epitopes which have a role in the life cycle of an Oomycota. These antibodies or fragments thereof may be inactivating in themselves or in combination with one or more other antibodies, or an anti-Oomycota substance (AOP), or in combination with a carrier, transmembrane domain or signal peptide. Importantly, plant pathogen resistance can be enhanced by the co-expression of multiple antibodies.

According to the present invention an anti-Oomycotic peptide or protein (AOP) has a detrimental effect on a Oomycota during its life cycle and/or an effect on the pathogen during plant infection, Oomycota growth or spreading. This includes anti-Oomycota substances that specifically kill an infected host cell and so limit the spread and development of a disease.

Examples of suitable anti-Oomycota substances include, but are not limited to, Cec, D4E1, GR7, Mag, MTK, or functionally active fragments thereof. Such AOPs according to the invention, or their fragments, can be used either alone or in any combination.

In principle all antibodies, proteins, peptides and enzymes that have a specificity and activity, that may or may not be enzymatic, which are able to interfere with Oomycota life cycles are suitable as part of the present constructs.

Genetic constructs according to the present invention may comprise the following or any combination of the following and may be encoded on one or more plasmids or clean DNA fragments: gene constructs may comprise a nucleotide sequence or nucleotide sequences encoding complete recombinant full-size antibodies, dimeric secretory IgA antibodies, multimeric IgM antibodies, the F(ab′)₂ fragment, the Fab-fragment, the Fv-fragment, single chain antibody fragments (scFvs), single binding domains (dAbs), a bivalent scFv (diabody), minibody, bispecific scFv antibodies where the antibody molecule recognizes two different epitopes that may come from the Oomycota or the host or both, triabodies and any other part of the antibody (molecular recognition units (MRUs)) which shows binding to the target epitopes. Genes encoding these suitable recombinant antibody fragment(s) may be derived from vertebrates, camelids, avian or pisces species or are synthetic.

In the constructs according to the invention, the antibody is fused to a complete sequence of an anti-Oomycota agent or a part thereof which still has activity, or which is still functionally active. The anti-Oomycota agent can be fused N- or C-terminal to the antibody or antibody fragment. Also, the chimeric protein may be encoded by nucleotide sequences on one or more constructs and may be assembled in vivo by the plant's or expression organism's protein assembly and translation machinery, respectively. The chimeric protein can also be obtained by biochemical assembly or in vitro or in vivo assembly of the chimeric fusion protein subunits using the cell's endogenous protein assembly and targeting machinery.

The antibody, antibodies or fragments thereof are fused directly to the anti-Oomycota agent or linked by a flexible spacer, which does not interfere with the structure or function of the two proteins. Such flexible linkers include copies of the (Glycine-Glycine-Glycine-Glycine-Serine)ₙ linker, where n is 1 to 4 or more copies of the linker unit, the Genex 212 and 218, 218* linker (the 218* linker contains one point mutation compared to the 218 linker leading to exchange of one tyrosine into a proline) and the flexible linker peptide of Trichoderma reesei cellobiohydrolase I (CBHI) (Turner et al. (1997) J Immunol Methods. 205:43-54).

The fusion construct comprising antibody, antibodies or fragments thereof, a linker and an anti-Oomycota agent or a part thereof or a fusion construct of antibody, antibodies or fragments thereof and anti-Oomycota agent or a part thereof can comprise an additional targeting sequence or a membrane anchor.

An example of a polynucleotide of the invention is a polynucleotide, wherein the antibody fragment is an scFv encoded by any of SEQ ID NO: 1 to SEQ ID NO: 9, or polynucleotides, wherein the AOP part is encoded by any of SEQ ID NO: 10 to SEQ ID NO: 14. An example of the before mentioned polynucleotides of the invention are polynucleotides, wherein an scFv encoding sequence is linked to an AOP encoding sequence via SEQ ID NO: 15 (coding for the (G₄S)₂ linker), and the scFv encoding sequence has the 5′ position with the AOP encoding portion in the 3′ position, or the AOP encoding portion has the 5′ position and the scFv encoding sequence has the 3′ position. Specific examples of polynucleotides according to the invention encoding a fusion protein as defined herein have a sequence selected from the group consisting of SEQ ID NO: 16 to SEQ ID NO: 60.

In the polynucleotide or vector, respectively, of the invention, the regulatory sequence is in particular selected from the group consisting of constitutive, chimeric, tissue specific and/or inducible synthetic and cryptic promoters.

A polynucleotide coding for a fusion protein of the invention preferably encodes a fusion protein with the general order [targeting sequence (Ts)-AOP-linker-antibody fragment-tag] wherein the antibody fragment is specific against Phytophthora, in particular wherein the Ts directs the fusion protein to the apoplast, the AOP is selected from Cec, D4E1, Mag and MTK, and the Phytophthora-specific antibody fragment tagged with c-myc and/or his₆. Examples of polynucleotides of the invention have a sequence according to any one of SEQ ID NO: 16 to SEQ ID NO: 60.

A further embodiment of the invention is a polynucleotide coding for a fusion protein of the invention wherein the above preferred order of the constituting elements is changed and/or the Ts, the linker (e.g. SEQ ID NO: 75; a nucleotide sequence coding therefore, see SEQ ID NO: 15) and/or the tag are missing.

Especially preferred constructs (fusion proteins, AA sequences and polynucleotide sequence coding therefore) and their components (antibody (fragment), AOP, linker) are listed in the following Table 1:

TABLE 1
Examples of fusion proteins and their components

| | | Amino | |
|---|---|---|---|
| | | acid | Nucleotide |
| | | sequence | sequence |
| | | according | according |
| | | to SEQ ID | to SEQ ID |
| Cconstruct/Component | Function | NO: | NO: * |
| scFvPi5 | Single-chain Fv ** | 1 | 61 |
| scFvPi33 | Single-chain Fv ** | 2 | 62 |
| scFvPi76.1 | Single-chain Fv ** | 3 | 63 |
| scFvPi76.2 | Single-chain Fv ** | 4 | 64 |
| scFvPi86 | Single-chain Fv ** | 5 | 65 |
| scFvPi88 | Single-chain Fv ** | 6 | 66 |
| scFvPi102.2 | Single-chain Fv ** | 7 | 67 |
| scFvPi129 | Single-chain Fv ** | 8 | 68 |
| scFvPi68 | Single-chain Fv ** | 9 | 69 |
| Cec | AOP | 10 | 70 |
| D4E1 | AOP | 11 | 71 |
| GR7 | AOP | 12 | 72 |
| Mag | AOP | 13 | 73 |
| MTK | AOP | 14 | 74 |
| (G₄S)₂ | AOP | 15 | 75 |
| Cec-(G₄S)₂-scFvPi5 | fusion protein ** | 16 | 76 |
| D4E1-(G₄S)₂-scFvPi5 | fusion protein ** | 17 | 77 |
| GR7-(G₄S)₂-scFvPi5 | fusion protein ** | 18 | 78 |
| Mag-(G₄S)₂-scFvPi5 | fusion protein ** | 19 | 79 |
| MTK-(G₄S)₂-scFvPi5 | fusion protein ** | 20 | 80 |
| Cec-(G₄S)₂-scFvPi33 | fusion protein ** | 21 | 81 |
| D4E1-(G₄S)₂-scFvPi33 | fusion protein ** | 22 | 82 |
| GR7-(G₄S)₂-scFvPi33 | fusion protein ** | 23 | 83 |
| Mag-(G₄S)₂-scFvPi33 | fusion protein ** | 24 | 84 |
| MTK-(G₄S)₂-scFvPi33 | fusion protein ** | 25 | 85 |
| Cec-(G₄S)₂-scFvPi76.1 | fusion protein ** | 26 | 86 |
| D4E1-(G₄S)₂-scFvPi76.1 | fusion protein ** | 27 | 87 |
| GR7-(G₄S)₂-scFvPi76.1 | fusion protein ** | 28 | 88 |
| Mag-(G₄S)₂-scFvPi76.1 | fusion protein ** | 29 | 89 |
| MTK-(G₄S)₂-scFvPi76.1 | fusion protein ** | 30 | 90 |
| Cec-(G₄S)₂-scFvPi76.2 | fusion protein ** | 31 | 91 |
| D4E1-(G₄S)₂-scFvPi76.2 | fusion protein ** | 32 | 92 |
| GR7-(G₄S)₂-scFvPi76.2 | fusion protein ** | 33 | 93 |
| Mag-(G₄S)₂-scFvPi76.2 | fusion protein ** | 34 | 94 |
| MTK-(G₄S)₂-scFvPi76.2 | fusion protein ** | 35 | 95 |
| Cec-(G₄S)₂-scFvPi86 | fusion protein ** | 36 | 96 |
| D4E1-(G₄S)₂-scFvPi86 | fusion protein ** | 37 | 97 |
| GR7-(G₄S)₂-scFvPi86 | fusion protein ** | 38 | 98 |
| Mag-(G₄S)₂-scFvPi86 | fusion protein ** | 39 | 99 |
| MTK-(G₄S)₂-scFvPi86 | fusion protein ** | 40 | 100 |
| Cec-G₄S)₂-scFvPi88 | fusion protein ** | 41 | 101 |
| D4E1-G₄S)₂-scFvPi88 | fusion protein ** | 42 | 102 |
| GR7-G₄S)₂-scFvPi88 | fusion protein ** | 43 | 103 |
| Mag-G₄S)₂-scFvPi88 | fusion protein ** | 44 | 104 |
| MTK-G₄S)₂-scFvPi88 | fusion protein ** | 45 | 105 |
| Cec-(G₄S)₂-scFvPi102.2 | fusion protein ** | 46 | 106 |
| D4E1-(G₄S)₂-scFvPi102.2 | fusion protein ** | 47 | 107 |
| GR7-(G₄S)₂-scFvPi102.2 | fusion protein ** | 48 | 108 |
| Mag-(G₄S)₂-scFvPi102.2 | fusion protein ** | 49 | 109 |
| MTK-(G₄S)₂-scFvPi102.2 | fusion protein ** | 50 | 110 |
| Cec-(G₄S)₂-scFvPi129 | fusion protein ** | 51 | 111 |
| D4E1-(G₄S)₂-scFvPi129 | fusion protein ** | 52 | 112 |
| GR7-(G₄S)₂-scFvPi129 | fusion protein ** | 53 | 113 |
| Mag-(G₄S)₂-scFvPi129 | fusion protein ** | 54 | 114 |
| MTK-(G₄S)₂-scFvPi129 | fusion protein ** | 55 | 115 |
| Cec-(G₄S)₂-scFvPi68 | fusion protein ** | 56 | 116 |
| D4E1-(G₄S)₂-scFvPi68 | fusion protein ** | 57 | 117 |
| GR7-(G₄S)₂-scFvPi68 | fusion protein ** | 58 | 118 |
| Mag-(G₄S)₂-scFvPi68 | fusion protein ** | 59 | 119 |
| MTK-(G₄S)₂-scFvPi68 | fusion protein ** | 60 | 120 |

* Note that all listed sequences of fusion proteins and components are displayed in the sequence listing without the C-terminal restriction site and without optional tag(s) (which can be included when expressed, e.g. in appropriate vectors such as pHENHi or pTRAkc).
** directed against Phytophthora infestans

In addition, the present invention relates to a kit as described above, e.g. in the form of a dip-stick-kit, an ELISA kit or protein chip comprising the above-described Ab, rAb, rAb fragments and their corresponding AOP fusion proteins. Said kit can also comprise the above described Ab, rAb, rAb fragments carrying at their C- or N-terminus a tag and/or are fused to a detection enzyme. Detection enzymes can be alkaline phosphatase and/or horse radish peroxidase. The kit of the invention may advantageously be used for carrying out diagnostic tests to detect Oomycota infections in crops or ornamental plants as well as harvestable materials thereof, or to detect the presence of one or more Oomycota in a collection of contaminated air.

The target pathogens of the present invention are plant Oomycota species. Oomycota plant pathogens cause devastating yield losses in crops and ornamental plants worldwide. The earliest food producers used mechanical means to control Oomycota outbreaks. Several Oomycota diseases could be overcome by the classic plant breeding. Moreover pesticides are used to control Oomycota pathogens, but they are very expensive and encompass health and environmental risks.

Genetic engineering is an alternative to chemical control, especially when there is no genetical resource for the breeding of new resistant varieties available. Several genes capable of controlling Oomycota plant pathogens have been inserted and expressed in plants. Most research efforts have been directed toward overexpression of the enzyme classes containing chitinases and glucanases (Benhamou (1995) Microsc. Res. Tech. 31: 63-78).

To date, antibody-based resistance has focused on pathogenic virus, bacteria, Ascomycota and nematodes, but the use of Ab, rAb, rAb fragments and their corresponding AOP fusions to protect plants against pathogenic Oomycota has not been investigated.

The figures show:

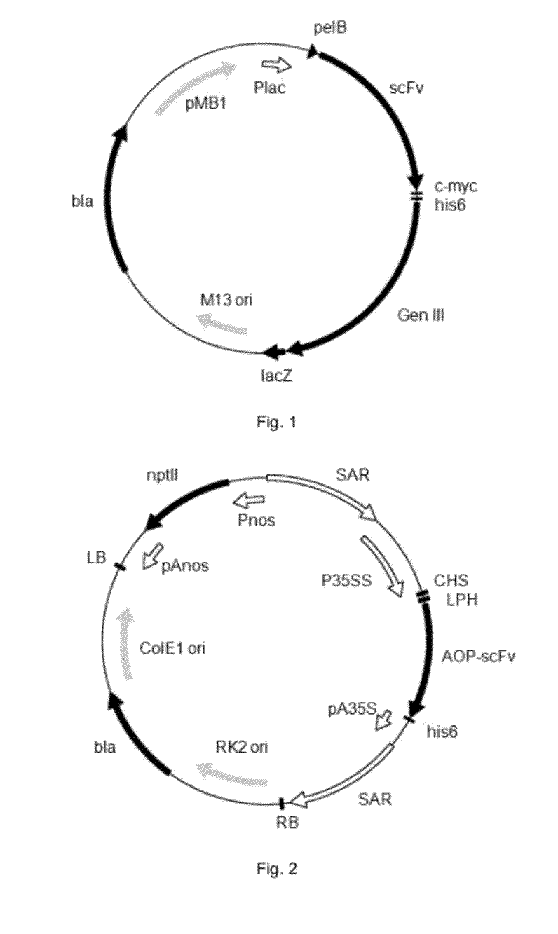

FIG. 1: shows a schematic representation of the vector pHENHi-scFv. rep (pMB1): origin of replication of the vector; Plac: lacZ promoter; c-myc: c-myc-tag for the detection of the recombinant protein; his6: his6-tag for the detection and purification of the recombinant protein; Gen III: Gen III protein of the envelope of phage M13; lacZ: 5′ sequence of the lacZ gene encoding the N-terminus of beta-galactosidase; M13 ori: origin of replication of the vector in M13 phages; bla: β-lactamase (resistance against ampicillin in E. coli).

FIG. 2: shows a schematic representation of the vector pTRAkc-AOP-scFv. RK2 ori: origin of replication of the vector in A. tumefaciens; bla: β-lactamase (ampicillin or carbenicillin, respectively, resistance in E. coli or A. tumefaciens, respectively); ColE1 ori: origin of replication of the vector in E. coli; LB and RB: left border and right border sequences of the nopalin-Ti-plasmid pTiT37; pAnos: termination and polyadenylation signal of the nopalin synthase gene (nos) from A. tumefaciens; nptII: neomycin phosphotransferase gene (resistance against kanamycin in plants); Pnos: promoter of the nos gene from A. tumefaciens; SAR: scaffold attachment region; P35SS: 35S promoter of CaMV with duplicated enhancer region; CHS: 5′-UTR of chalkonsynthase from Petroselium; LPH: codon-optimised version of the murine signal peptide of the heavy chain of anti-TMV mAb24 (Vaquero et al. Proc Natl Acad Sci USA. 1999 Sep. 28; 96(20):11128-33.); AOP-scFv: sequence encoding the construct of anti-Oomycota peptide and scFv; his6: his₆-tag for the detection and purification of the recombinant protein; pA35S: 3′UTR of the CaMV 35S gene.

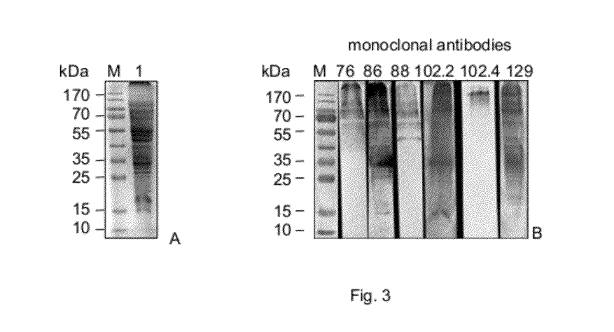

FIG. 3. shows the results of SDS-PAGE separations of P. infestans cell wall fragments (A) and the corresponding immunoblot analysis using P. infestans-specific monoclonal antibodies (B). Cell wall fragments of P. infestans (200 μg DW) were separated electrophoretically on 12% SDS-polyacrylamide gels (A), transferred to nitrocellulose and incubated with 1 ml supernatant of cultures of the selected monoclonal hybridoma cell lines. Bound monoclonal antibodies were detected by GAM^{AP} Fc (1:5000) or GAM^{HPP} IgM (1:5000), respectively, followed by visualisation through NBT/BCIP or 4-chloro-1-naphthol staining (B). M: Prestained Protein Marker (Fermentas); 1: P. infestans cell wall fragments (200 μg DW).

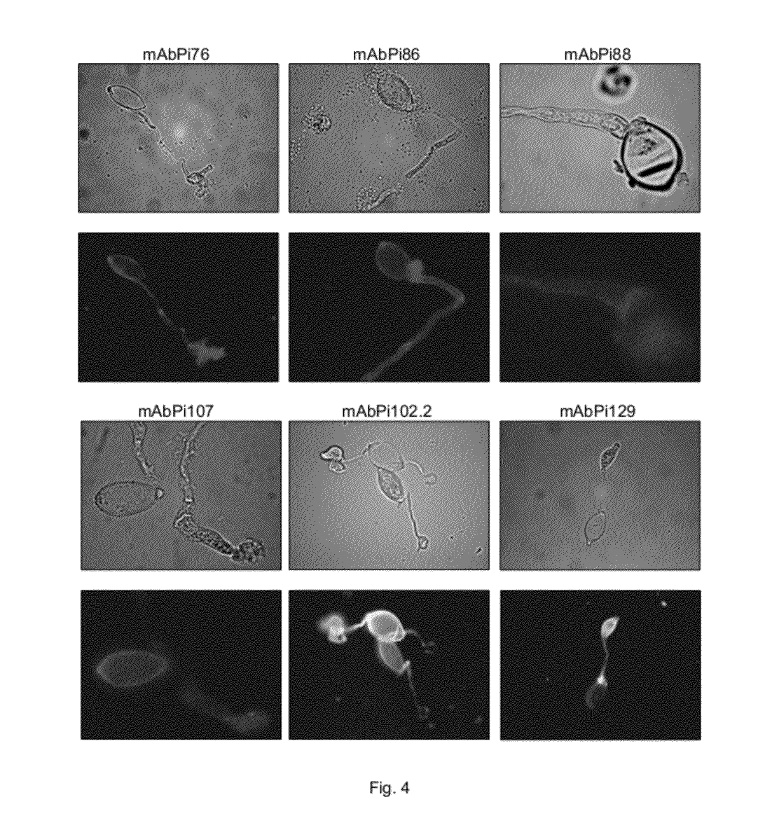

FIG. 4: shows photographs of immunofluorescence microscopic analysis of the binding of monoclonal antibodies to germinated sporangia of P. infestans. Germinated sporangia of P. infestans were immobilised on poly-L-lysine-coated coverslips and incubated with monoclonal antibodies (0.2 ml culture supernatant). Bound antibodies were detected using GAM^{FITC} H+L or GAM^{Alexa} IgM (1:500). Antigen-antibody complexes were visualised by fluorescence microscopy (lines 2 and 4). Light microscopic photographs of the corresponding experiment are shown for comparison (lines 1 and 3). Negative controls were carried out with MAH Fc and IgM κ. Negative controls showed no detectable fluorescence signal (not shown).

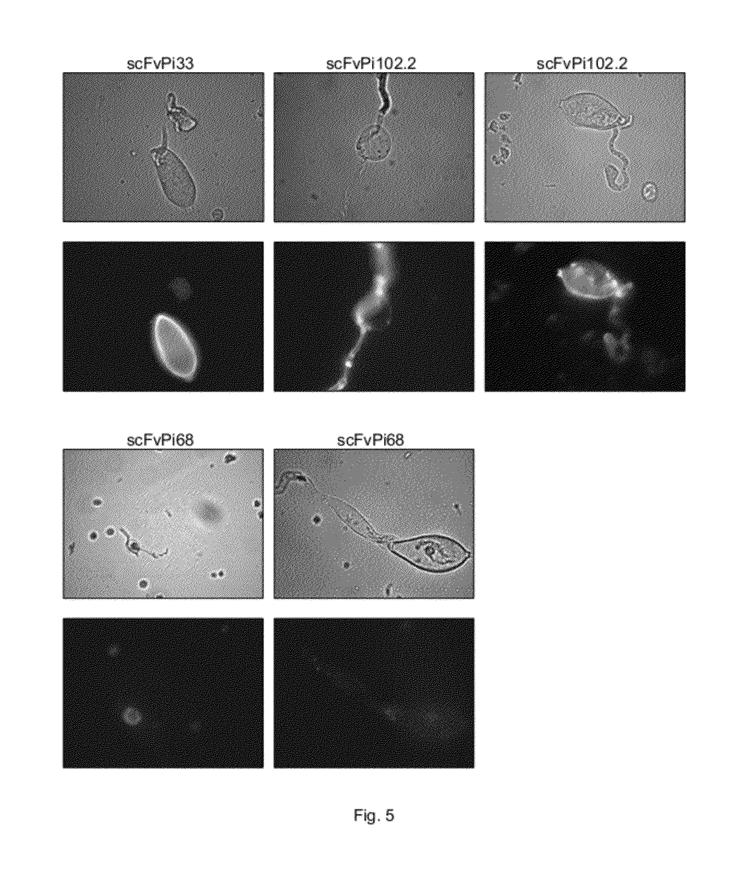

FIG. 5: shows photographs of immunofluorescence microscopic analysis of the binding of scFv to germinated sporangia and zoospores of P. infestans. Germinated sporangia and zoospores of P. infestans were immobilised on poly-L-lysine-coated coverslips and incubated with purified scFv. Bound scFv were detected using α-c-myc mAb (1:500) and GAM^{FITC} H+L. Antibody-antigen complexes were visualised by fluorescence microscopy (photographs in lines 2 and 4). Light microscopic photographs of the same specimen are shown for comparison (lines 1 and 3). Negative controls were carried out with scFvODC3/2 (Nölke 2002 PhD thesis RWTH Aachen). No fluorescence signal was detected in the negative controls (data not shown).

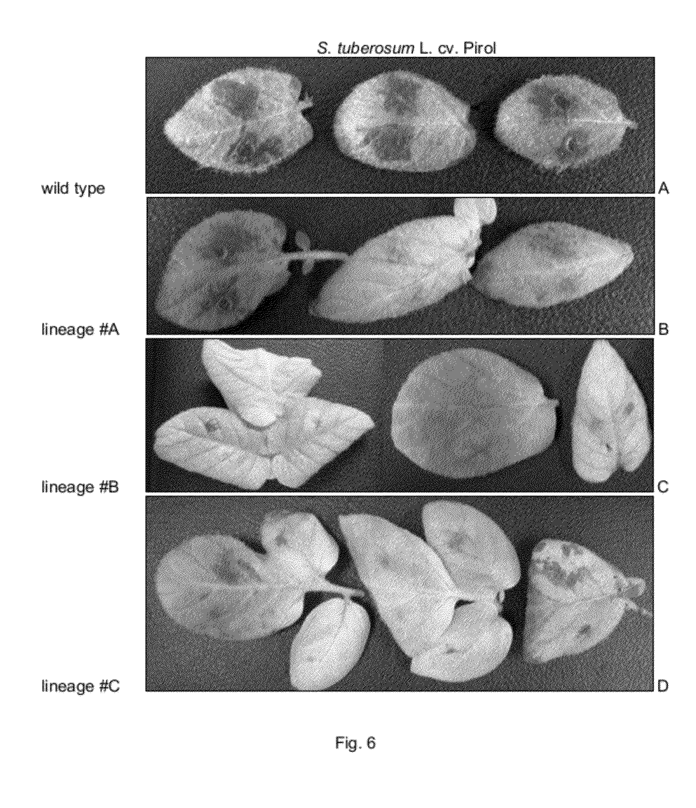

FIG. 6 shows photographs of potato leafs demonstrating resistance of transgenic S. tuberosum L. cv Pirol against P. infestans. Leafs of transgenic potato plants of the variety Pirol carrying the transgene GR7-scFvPi102.2 were taken from the second third of the plants and placed into petri dishes on wet paper with the abaxial side facing up. The inoculation with P. infestans was carried out by placing 5 μl of a zoospore solution (1×10⁴ sporangia/ml) on two locations of a leaf. Photographs were taken on day 5 after inoculation. A: leafs of a wild type plant inoculated with P. infestans. B: leafs of lineage #A showing reduced sporulation in comparison to the wild type. C: leafs of lineage #B one of which showed a reduced sporulation in comparison to the wild type, and two of which showed no visible sporangiophores on the leaf surface. D: leafs of the lineage #C showed no visible sporangiophores.

The present invention is further illustrated by the following non-limited examples:

### EXAMPLES

### Example 1

### Generation of Phytophthora infestans-Specific scFvs by Hybridoma Technology

1. Cell wall fragments were prepared from P. infestans.

2. Mice were immunized with the cell wall fragments.

3. Spleen cells from immunized mice were isolated and hybridomas were generated. Several limiting dilution steps were performed to isolate hybridoma cell lines that secrete antibodies specifically recognizing P. infestans antigens.

4. mRNA from selected hybridoma cell lines was isolated and cDNA generated using reverse transcriptase. cDNA sequences encoding the antibody variable heavy and light chains (VH and VL) were amplified by PCR and cloned into the pHENHi and pTRAkc vector.

5. The final scFv constructs were used for bacterial and plant expression.

### Example 2

### Generation of Phytophthora infestans-Specific scFvs by Phage Display

1. Cell wall fragments were prepared from P. infestans.

2. Chicken and mice were immunized with the cell wall fragments.

3. mRNA from chicken and mice spleen cells was isolated and cDNA generated using reverse transcriptase. Variable domains of heavy and light chains (VH and VL) were amplified by PCR and cloned via unique restriction sites separately into the phage display vector pHENHi to generate a VH and a VL library. pHENHi contains a pelB signal sequence for targeting of recombinant proteins to the bacterial periplasm and a C-terminal c-myc- and his-tag. Subsequently, VL fragments were cut out from the VL library and ligated into the VH library to assemble the scFv cDNA whereas VH and VL cDNAs were connected by a linker peptide.

4. Phage libraries derived from the different scFv libraries (step 3) were generated and specific scFv fragments were identified by library panning using germinated sporangia and cell wall fragments of P. infestans. After each panning round eluted phages were used for infection of E. coli and the new phage libraries were prepared for the next round of panning. After three rounds of panning the best binders were selected by ELISA.

5. The final scFvs were expressed in bacteria and plants and tested against P. infestans antigens.

### Example 3

### Characterization of mAbs and scFvs

1. mAbPi76, mAbPi86, mAbPi88, mAbPi102.2, mAbPi129 were produced in hybridoma cell culture and mAb-containing supernatant was used to characterise the mAbs by immunoblot, ELISA and immunofluorescence microscopy.

2. ScFvPi5, scFvPi33, scFvPi68, scFvPi86, scFvPi88, scFvPi102.2, scFvPi129 integrated in pHENHi (FIG. 1) were bacterially expressed, some purified by IMAC and characterized by Immunoblot, ELISA and immunofluorescence microscopy.

3. Immunoblot (FIG. 3), ELISA and immunofluorescence microscopy (FIG. 4) confirmed binding of mAbPi76, mAbPi86, mAbPi88, mAbPi102.2, mAbPi129 to P. infestans cell wall fragments as well as to the surface of sporangia and germ tubes of the intact germinated pathogen.

4. Immunoblot confirmed binding of scFvPi33 and scFvPi68 to P. infestans mycelium preparations containing cytosolic components as well as binding of scFvPi33 to P. infestans cell wall fragments. Binding of scFvPi5 could not be verified by immunoblot.

5. ELISA confirmed binding of scFvPi5, scFvPi33, scFvPi68, scFvPi86, scFvPi88, scFvPi102.2 and scFvPi129 to P. infestans cell wall fragments.

6. Immunofluorescence microscopy showed binding of scFvPi33 mainly to the surface of P. infestans sporangia, binding of scFvPi102.2 to the surface of sporangia, zoospores and germ tubes of P. infestans and binding of scFvPi68 to zoospores and intracellular components of P. infestans (FIG. 5).

### Example 4

### Construction of AOP and AOP-scFv Fusions

1. AOP-scFv cDNAs of D4E1-scFvPi102.2 and GR7-scFvPi102.2 were cloned into the pHENHi and pTRAkc vectors

2. Bacterially expressed and transiently in N. tabacum produced fusion proteins were purified by IMAC. Binding of bacterially expressed Mag-scFvPi102.2 and transiently expressed D4E1-scFvPi102.2 as well as GR7-scFvPi102.2 to P. infestans cell wall fragments was verified by ELISA.

### Example 5

### Stable Transformation of S. tuberosum and Resistance Tests

1. AOP-scFv cDNAs of D4E1-scFvPi102.2 and GR7-scFvPi102.2 were integrated into the plant expression vector pTRAkc (FIG. 2).

2. S. tuberosum leaves were stable transformed using recombinant A. tumefaciens.

3. Regenerated plants were analyzed by PCR to verify the integration of the transgene into the plant genome.

4. Detached leaves of transgenic plants were infected with zoospores of P. infestans and analyzed according to the density of sporangiophores on the leave surface five days post inoculation. Transgenic plants completely free of sporangiophores were observed for both transgenes and regarded as resistant (FIG. 6).

1. A fusion protein comprising at least one anti-Oomycotic protein or peptide (AOP) linked to an antibody or fragment thereof specifically recognising an epitope of an Oomycota.

2. The fusion protein of claim 1 wherein the AOP is selected from the group consisting of Cec, D4E1, GR7, Mag and MTK.

3. The fusion protein of claim 1 or 2 wherein the antibody or fragment thereof specifically recognises an epitope of Phytophthora ssp., Pythium ssp., Peronospora ssp. and Pseudoperonospora ssp., preferably Phytophthora infestans, Phytophthora capsici, Phytophthora cactorum, Phytophthora cannamoni or Phytophthora nicotianae.

4. The fusion protein according to any one of the preceding claims wherein the antibody or fragment thereof is a F(ab′)2 fragment, Fab fragment, scFv, bi-specific scFv, tri-specific scFv, diabody, single domain antibody (dAb), minibody or molecular recognition unit (MRU).

5. The fusion protein of claim 4 wherein the antibody fragment is a scFv having a sequence selected from the group consisting of SEQ ID NO: 61 to SEQ ID NO: 69.

6. The fusion protein according to any one of the preceding claims further comprising at least one N-terminal and/or C-terminal tag at least facilitating the detection and/or purification of the fusion protein.

7. The fusion protein of claim 6 wherein the tag is selected from the group consisting of c-myc, his₆, his₅, tag54, FLAG, HA, HSV-, T7, S, strep and E-tag.

8. The fusion protein according to any one of the preceding claims further comprising a cellular targeting sequence.

9. The fusion protein of claim 8 wherein the cellular targeting sequence is a sequence for secretion or location of the fusion protein to cell compartments or organelles, preferably the apoplast, the vacuole, intra- and/or exterior membranes or the ER lumen.

10. The fusion protein according to any one of the preceding claims wherein the AOP and the antibody or fragment thereof are linked by a peptide linker.

11. The fusion protein of claim 10 comprising a sequence selected from the group consisting of SEQ ID NO: 76 to SEQ ID NO: 120.

12. A polynucleotide comprising a sequence encoding the fusion protein according to any one of the preceding claims.

13. The polynucleotide of claim 12 wherein the sequence is optimized for expression in a host cell.

14. A vector containing the polynucleotide of claim 12 or 13 within an expression cassette.

15. The vector of claim 14 wherein the expression cassette is operatively linked to one or more regulatory sequence(s) allowing the expression of the fusion protein, preferably in plants, plant organs, plant tissues and/or plant cells.

16. A host cell comprising the polynucleotide of claim 12 or 13 and/or the vector of claim 14 or 15.

17. A method for the production of the fusion protein according to any one of claims 1 to 11 comprising the steps:
(a) culturing host cells of claim 16 in a culture medium under conditions allowing the expression of the fusion protein according to any one of claims 1 to 11; and
(b) recovering the fusion protein from the medium and/or the host cells.

18. A method for the production of a Oomycota-resistant plant, plant cell or plant tissue comprising the step of introducing the polynucleotide of claim 12 or 13 into the genome of the plant, plant cell or plant tissue.

19. A transgenic plant or plant tissue transformed with the polynucleotide of claim 12 or 13.

20. Harvestable parts and propagation materials derived from the transgenic plant of claim 19.

21. Use of the fusion protein according to any one of claims 1 to 11, the polynucleotide of claim 12 or 13 and/or the vector of claim 14 or 15 for the protection of a plant against the action of Oomycota.

22. A kit comprising the fusion protein according to any one of claims 1 to 11 and/or the polynucleotide of claim 12 or 13 and/or the vector of claim 14 or 15 and/or the host cell of claim 16 together with means for the detection of said fusion protein, polynucleotide, vector and/or host cell.


SEQUENCE LISTING (120 sequences)

SEQ ID NO: 1 - DNA, 756 bp, Artificial
  coding sequence for scFvPi5 with specificity against Phytophthora infestans; originates from Mus musculus
atggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 2 - DNA, 771 bp, Artificial
  coding sequence for scFvPi33 with specificity against Phytophthora infestans; originates from Mus musculus
atggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 3 - DNA, 753 bp, Artificial
  coding sequence for scFvPi76.1 with specificity against Phytophthora infestans; originates from Mus musculus
atgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 4 - DNA, 753 bp, Artificial
  coding sequence for scFvPi76.2 with specificity against Phytophthora infestans; originates from Mus musculus
atgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 5 - DNA, 756 bp, Artificial
  coding sequence for scFvPi86 with specificity against Phytophthora infestans; originates from Mus musculus
atggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 6 - DNA, 786 bp, Artificial
  coding sequence for scFvPi88 with specificity against Phytophthora infestans; originates from Mus musculus
atgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 7 - DNA, 756 bp, Artificial
  coding sequence for scFvPi102.2 with specificity against Phytophthora infestans; originates from Mus musculus
atggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 8 - DNA, 738 bp, Artificial
  coding seqeuence for scFvPi129 with specificity against Phytophthora infestans; originates from Mus musculus
atggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 9 - DNA, 780 bp, Artificial
  coding sequence  for scFvPi68 with specificity against Phytophthora infestans; originates from Gallus gallus
atggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 10 - DNA, 42 bp, Artificial
  coding sequence for Cec; originates from Hyalophora cecropia; synthetic Cecropin A-derived peptide
atggctaagtggaagttgtttaagaagattttgaaggttttg

SEQ ID NO: 11 - DNA, 57 bp, Artificial
  coding sequence for D4E1; synthetic peptide
atgggatttaagttgagagctaagattaaggttagattgagagctaagattaagttg

SEQ ID NO: 12 - DNA, 66 bp, Artificial
  coding sequence for GR7; synthetic peptide
atgggaatggcttctagagctgctagattggctgctagattggctaggctcgctctcagggcgctc

SEQ ID NO: 13 - DNA, 75 bp, Artificial
  coding sequence for Mag; originates from Xenopus laevis Magainin 1 peptide
atgggaggtattggtaagtttttgcattctgctggtaagtttggtaaggcttttgttggtgaaattatgaagtct

SEQ ID NO: 14 - DNA, 84 bp, Artificial
  coding sequence for MTK (metchnikowin); codon- optimised for dicotyles
atgggacatagaagagagggacctatttttgatactagaccttctccattcaatcctaatcaacctagacctggacctatttat

SEQ ID NO: 15 - DNA, 30 bp, Artificial
  coding sequence for (G4S)2 linker
ggtggaggcggatctggtggcggtggaagc

SEQ ID NO: 16 - DNA, 849 bp, Artificial
  coding sequence for Cec-(G4S)2-scFvPi5; fusion protein comprising Cec-(G4S)2-scFvPi5
atggctaagtggaagttgtttaagaagattttgaaggttttggtcgacggtggaggcggatctggtggcggtggaagcgcggcccagccggccatggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 17 - DNA, 864 bp, Artificial
  coding sequence for D4E1-(G4S)2-scFvPi5; fusion protein comprising D4E1-(G4S)2-scFvPi5
atgggatttaagttgagagctaagattaaggttagattgagagctaagattaagttggtcgacggtggaggcggatctggtggcggtggaagcgcggcccagccggccatggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 18 - DNA, 873 bp, Artificial
  coding sequence for GR7-(G4S)2-scFvPi5; fusion protein comprising GR7-(G4S)2-scFvPi5
atgggaatggcttctagagctgctagattggctgctagattggctaggctcgctctcagggcgctcgtcgacggtggaggcggatctggtggcggtggaagcgcggcccagccggccatggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 19 - DNA, 882 bp, Artificial
  coding sequence for Mag-(G4S)2-scFvPi5; fusion protein comprising Mag-(G4S)2-scFvPi5
atgggaggtattggtaagtttttgcattctgctggtaagtttggtaaggcttttgttggtgaaattatgaagtctgtcgacggtggaggcggatctggtggcggtggaagcgcggcccagccggccatggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 20 - DNA, 891 bp, Artificial
  coding sequence for MTK-(G4S)2-scFvPi5; fusion protein comprising MTK-(G4S)2-scFvPi5
atgggacatagaagagagggacctatttttgatactagaccttctccattcaatcctaatcaacctagacctggacctatttatgtcgacggtggaggcggatctggtggcggtggaagcgcggcccagccggccatggccgaggttcagctgcagcagtctggacctgagctggtgaagcctggagcttcaatgaagatatcctgcaaggcttctggttactcattcactggctacaccatgaactggatgaagcagagccatggaaagagccttgagtggattggacttattaatccttacaatggtggttctagctacaaccagaagttcaagggcaaggccacattaactgtagacaagtcatccagcactgcctacatggagctcctcagtctgacatctgaggactctgcagtctattactgtgcaagagggagtactacgggcccgtacttcgatgtctggggcgcagggaccacggtcaccgtctcctcaggctccacctcaggctccggtaaacctggcccaggggagggatcaactaagggcgcgcctgatatccagatgacccagtctacatcctccctgtctgcctctctgggagacagagtcaccatcaattgcagggcaagtcaggacattagcaattatttaaactggtatcagcagaaaccagacggaactgttaaactcctgatctactacacatcaagattacactcaggagtcccatcaaggttcagtggcagtgggtctggaacagattattctctcaccattaacaacctggagcaagaagatattgccacttacttttgccaacagggtcatacgcttccgtacacgttcggaggggggaccaaattggaactgaaacgtgctgtcgac

SEQ ID NO: 21 - DNA, 864 bp, Artificial
  coding sequence for Cec-(G4S)2-scFvPi33; fusion protein comprising Cec-(G4S)2-scFvPi33
atggctaagtggaagttgtttaagaagattttgaaggttttggtcgacggtggaggcggatctggtggcggtggaagcgcggcccagccggccatggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 22 - DNA, 879 bp, Artificial
  coding sequence for D4E1-(G4S)2-scFvPi33; fusion protein comprising D4E1-(G4S)2-scFvPi33
atgggatttaagttgagagctaagattaaggttagattgagagctaagattaagttggtcgacggtggaggcggatctggtggcggtggaagcgcggcccagccggccatggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 23 - DNA, 888 bp, Artificial
  coding sequence for GR7-(G4S)2-scFvPi33; fusion protein comprising GR7-(G4S)2-scFvPi33
atgggaatggcttctagagctgctagattggctgctagattggctaggctcgctctcagggcgctcgtcgacggtggaggcggatctggtggcggtggaagcgcggcccagccggccatggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 24 - DNA, 897 bp, Artificial
  coding sequence for Mag-(G4S)2-scFvPi33; fusion protein comprising Mag-(G4S)2-scFvPi33
atgggaggtattggtaagtttttgcattctgctggtaagtttggtaaggcttttgttggtgaaattatgaagtctgtcgacggtggaggcggatctggtggcggtggaagcgcggcccagccggccatggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 25 - DNA, 906 bp, Artificial
  coding sequence for MTK-(G4S)2-scFvPi33; fusion protein comprising MTK-(G4S)2-scFvPi33
atgggacatagaagagagggacctatttttgatactagaccttctccattcaatcctaatcaacctagacctggacctatttatgtcgacggtggaggcggatctggtggcggtggaagcgcggcccagccggccatggccgaggttcagcttcagcagtctggacctgagctggtaaagcctggggcttcagtgaagatgtcctgcaagacttctggatacacattcactaactatattatgcactgggtgaagcagcagccagggcagggccttgagtggattggatatattaatccttacaatgatgctactaagtacaatgagaagttcaaaggcaaggccacactgacttcagacagatcctccagcacagcctacatggagctcagcagcctgacctctgaggactctgcggtctattactgtgcaagagttggtgatgtcagtgggtttgcttactggggccaagggaccctggtcaccgtctcctcaggctccacctcaggctccggtaaacctggcccaggggagggatcaactaagggcgcgcctgacattgtgctgtctcagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagttggagataaaacgtgctgtcgac

SEQ ID NO: 26 - DNA, 846 bp, Artificial
  coding sequence for Cec-(G4S)2-scFvPi76.1; fusion protein comprising Cec-(G4S)2-scFvPi76.1
atggctaagtggaagttgtttaagaagattttgaaggttttggtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 27 - DNA, 861 bp, Artificial
  coding sequence for D4E1-(G4S)2-scFvPi76.1; fusion protein comprising D4E1-(G4S)2-scFvPi76.1
atgggatttaagttgagagctaagattaaggttagattgagagctaagattaagttggtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 28 - DNA, 870 bp, Artificial
  coding sequence for GR7-(G4S)2-scFvPi76.1; fusion protein comprising GR7-(G4S)2-scFvPi76.1
atgggaatggcttctagagctgctagattggctgctagattggctaggctcgctctcagggcgctcgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 29 - DNA, 879 bp, Artificial
  coding sequence for Mag-(G4S)2-scFvPi76.1; fusion protein comprising Mag-(G4S)2-scFvPi76.1
atgggaggtattggtaagtttttgcattctgctggtaagtttggtaaggcttttgttggtgaaattatgaagtctgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 30 - DNA, 888 bp, Artificial
  coding sequence for MTK-(G4S)2-scFvPi76.1; fusion protein comprising MTK-(G4S)2-scFvPi76.1
atgggacatagaagagagggacctatttttgatactagaccttctccattcaatcctaatcaacctagacctggacctatttatgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacaatcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattatgttttcggcagtggaaccaaggtcactgtcctaggtcagcccaag

SEQ ID NO: 31 - DNA, 846 bp, Artificial
  coding sequence for Cec-(G4S)2-scFvPi76.2; fusion protein comprising Cec-(G4S)2-scFvPi76.2
atggctaagtggaagttgtttaagaagattttgaaggttttggtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 32 - DNA, 861 bp, Artificial
  coding sequence for D4E1-(G4S)2-scFvPi76.2; fusion protein comprising D4E1-(G4S)2-scFvPi76.2
atgggatttaagttgagagctaagattaaggttagattgagagctaagattaagttggtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 33 - DNA, 870 bp, Artificial
  coding sequence for GR7-(G4S)2-scFvPi76.2; fusion protein comprising GR7-(G4S)2-scFvPi76.2
atgggaatggcttctagagctgctagattggctgctagattggctaggctcgctctcagggcgctcgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 34 - DNA, 879 bp, Artificial
  coding sequence for Mag-(G4S)2-scFvPi76.2; fusion protein comprising Mag-(G4S)2-scFvPi76.2
atgggaggtattggtaagtttttgcattctgctggtaagtttggtaaggcttttgttggtgaaattatgaagtctgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 35 - DNA, 888 bp, Artificial
  coding sequence for MTK-(G4S)2-scFvPi76.2; fusion protein comprising MTK-(G4S)2-scFvPi76.2
atgggacatagaagagagggacctatttttgatactagaccttctccattcaatcctaatcaacctagacctggacctatttatgtcgacggtggaggcggatctggtggcggtggaagcgcggcccagccggccatgggtgaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaagccagatagcagtacgataaactatacgccatctctaaaggataaattcatcatctccagagacaacgccaaaaattcgctgtacctgcaaatgagcagagtgagatctgaggacacagccctttattactgtgcaagacggggtggttactttgcttactggggccaagggactctggtcaccgtctcctcaggctccacctcaggctccggtaaacctggcccaggggagggatcaactaagggcgcgccccaggctgttgtgactcaggaatctgcactcaccacatcacctggtggaacagtcatactcacttgtcgctcaagtactggggctgtgacaactagtaacaatgccaactgggtccaagaaaaaccagatcatttattcactggtctaataggtggtaccagcaaccgagctccaggtgttcctgtcagattctcaggctccctgattggagacaaggctgccctcaccatcacaggggcacagactgaggatgatgcaatgtatttctgtgctctatggtacagcacccattatgttttcggcggtggaaccaaggtcactgtcctaggtcagcccaag

SEQ ID NO: 36 - DNA, 849 bp, Artificial
  coding sequence for Cec-(G4S)2-scFvPi86; fusion protein comprising Cec-(G4S)2-scFvPi86
atggctaagtggaagttgtttaagaagattttgaaggttttggtcgacggtggaggcggatctggtggcggtggaagcgcggcccagccggccatggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 37 - DNA, 864 bp, Artificial
  coding sequence for D4E1-(G4S)2-scFvPi86; fusion protein comprising D4E1-(G4S)2-scFvPi86
atgggatttaagttgagagctaagattaaggttagattgagagctaagattaagttggtcgacggtggaggcggatctggtggcggtggaagcgcggcccagccggccatggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 38 - DNA, 873 bp, Artificial
  coding sequence for GR7-(G4S)2-scFvPi86; fusion protein comprising GR7-(G4S)2-scFvPi86
atgggaatggcttctagagctgctagattggctgctagattggctaggctcgctctcagggcgctcgtcgacggtggaggcggatctggtggcggtggaagcgcggcccagccggccatggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 39 - DNA, 882 bp, Artificial
  coding sequence for Mag-(G4S)2-scFvPi86; fusion protein comprising Mag-(G4S)2-scFvPi86
atgggaggtattggtaagtttttgcattctgctggtaagtttggtaaggcttttgttggtgaaattatgaagtctgtcgacggtggaggcggatctggtggcggtggaagcgcggcccagccggccatggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 40 - DNA, 891 bp, Artificial
  coding sequence for MTK-(G4S)2-scFvPi86; fusion protein comprising MTK-(G4S)2-scFvPi86
atgggacatagaagagagggacctatttttgatactagaccttctccattcaatcctaatcaacctagacctggacctatttatgtcgacggtggaggcggatctggtggcggtggaagcgcggcccagccggccatggcccaggtccaactgcagcagcctgggtctgtgctggtgaggcctggagcttcagtgaagctgtcctgcaaggcttctggctacaccttcaccagctcctggatgcactgggcgaagcagaggcctggacaaggccttgagtggattggagagattcatcctaatagtggtgaaagtcaccacaatgagaagttcaagggcaaggccacactgactgtagacacatccaccagcacagcctacgtggatctcagcagcctgacatctgaggactctgcggtttattactgtgtaagagggcagctcgggccttcttactggggccaagggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagtaactatgccaactgggtccaagaaaaaccagatcattcattcactagtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 41 - DNA, 879 bp, Artificial
  coding sequence for Cec-(G4S)2-scFvPi88; fusion protein comprising Cec-(G4S)2-scFvPi88
atggctaagtggaagttgtttaagaagattttgaaggttttggtcgacggtggaggcggatctggtggcggtggaagcgcggcccagccggccatgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 42 - DNA, 894 bp, Artificial
  coding sequence for D4E1-(G4S)2-scFvPi88; fusion protein comprising D4E1-(G4S)2-scFvPi88
atgggatttaagttgagagctaagattaaggttagattgagagctaagattaagttggtcgacggtggaggcggatctggtggcggtggaagcgcggcccagccggccatgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 43 - DNA, 903 bp, Artificial
  coding sequence for GR7-(G4S)2-scFvPi88; fusion protein comprising GR7-(G4S)2-scFvPi88
atgggaatggcttctagagctgctagattggctgctagattggctaggctcgctctcagggcgctcgtcgacggtggaggcggatctggtggcggtggaagcgcggcccagccggccatgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 44 - DNA, 912 bp, Artificial
  coding sequence for Mag-(G4S)2-scFvPi88; fusion protein comprising Mag-(G4S)2-scFvPi88
atgggaggtattggtaagtttttgcattctgctggtaagtttggtaaggcttttgttggtgaaattatgaagtctgtcgacggtggaggcggatctggtggcggtggaagcgcggcccagccggccatgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 45 - DNA, 921 bp, Artificial
  coding sequence for MTK-(G4S)2-scFvPi88; fusion protein comprising MTK-(G4S)2-scFvPi88
atgggacatagaagagagggacctatttttgatactagaccttctccattcaatcctaatcaacctagacctggacctatttatgtcgacggtggaggcggatctggtggcggtggaagcgcggcccagccggccatgggtgaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcttcgtctccagagacacttcccaaagcatcctctaccttcagatgaatgccctgagagctgaggacactgccatttattactgtgcaagagattactacggtagtagctactggtacttcgatgtctggggcgcagggaccacggtcaccgtctcctcaggctccacctcaggctccggtaaacctggcccaggggagggatcaactaagggcgcgccagacattgtgatgactcagtctccaactttccttgctgtgacagcaagtaagaaggtcaccattagttgcacggccagtgagagcctttattcaagcaaacacaaggtgcactacttggcttggtaccagaagaaaccagagcaatctcctaaactgctgatatacggggcatccaaccgatacattggggtccctgatcgcttcacaggcagtggatctgggacagatttcactctgaccatcagcagtgtacaggttgaagacctcacacattattactgtgcacagttttacagatatccgctcacgttcggtgctgggaccaagctggagctgaaacgggctgatgct

SEQ ID NO: 46 - DNA, 849 bp, Artificial
  coding sequence for Cec-(G4S)2-scFvPi102.2; fusion protein comprising Cec-(G4S)2-scFvPi102.2
atggctaagtggaagttgtttaagaagattttgaaggttttggtcgacggtggaggcggatctggtggcggtggaagcgcggcccagccggccatggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 47 - DNA, 864 bp, Artificial
  coding sequence for D4E1-(G4S)2-scFvPi102.2; fusion protein comprising D4E1-(G4S)2-scFvPi102.2
atgggatttaagttgagagctaagattaaggttagattgagagctaagattaagttggtcgacggtggaggcggatctggtggcggtggaagcgcggcccagccggccatggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 48 - DNA, 873 bp, Artificial
  coding sequence for GR7-(G4S)2-scFvPi102.2; fusion protein comprising GR7-(G4S)2-scFvPi102.2
atgggaatggcttctagagctgctagattggctgctagattggctaggctcgctctcagggcgctcgtcgacggtggaggcggatctggtggcggtggaagcgcggcccagccggccatggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 49 - DNA, 882 bp, Artificial
  coding sequence for Mag-(G4S)2-scFvPi102.2; fusion protein comprising Mag-(G4S)2-scFvPi102.2
atgggaggtattggtaagtttttgcattctgctggtaagtttggtaaggcttttgttggtgaaattatgaagtctgtcgacggtggaggcggatctggtggcggtggaagcgcggcccagccggccatggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 50 - DNA, 891 bp, Artificial
  coding sequence for MTK-(G4S)2-scFvPi102.2; fusion protein comprising MTK-(G4S)2-scFvPi102.2
atgggacatagaagagagggacctatttttgatactagaccttctccattcaatcctaatcaacctagacctggacctatttatgtcgacggtggaggcggatctggtggcggtggaagcgcggcccagccggccatggcccaggtccaactgcagcagcctgggtttgtattggtgaggcctggagcttcagtgaagctgtcctgcaaggcttctggctactccttcaccagcacctggatgcactgggcgaaacagaggcctggacaaggccttgagtggattggcgagattcatcctaatagtggtgatactcactacaatgagaagttcaagggcaaggccacactgactgtagacacatcctccagcacagcctacgtggatctcagcagcctgacatctgaggactctgcggtctattactgtgtaagagggcccctcgggccttcttactggggccaggggactctggtcaccgtctcctcaggctccacctcaggctccggtaaacctggcccaggggagggatcaactaagggcgcgccccaggctgttgtgactcaggaatctgcactcaccacatcacctggtgaaacagtcacactcacttgtcgctcaagtactggggctgttacaactagcaactatgccaactgggtccaagaaaaaccagatcatttattcactggtctaataagtggtaccaacaaccgagctccaggtgttcctgccagattctcaggctccctgattggagacaaggctgccctcaccatcacaggggcacagactgaggatgaggcaatatatttctgtgctctatggtacagcaaccatttctgggtgttcggtggaggaaccaaactgactgtcctaggccagcccaag

SEQ ID NO: 51 - DNA, 831 bp, Artificial
  coding sequence for Cec-(G4S)2-scFvPi129; fusion protein comprising Cec-(G4S)2-scFvPi129
atggctaagtggaagttgtttaagaagattttgaaggttttggtcgacggtggaggcggatctggtggcggtggaagcgcggcccagccggccatggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 52 - DNA, 846 bp, Artificial
  coding sequence for D4E1-(G4S)2-scFvPi129; fusion protein comprising D4E1-(G4S)2-scFvPi129
atgggatttaagttgagagctaagattaaggttagattgagagctaagattaagttggtcgacggtggaggcggatctggtggcggtggaagcgcggcccagccggccatggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 53 - DNA, 855 bp, Artificial
  coding sequence for GR7-(G4S)2-scFvPi129; fusion protein comprising GR7-(G4S)2-scFvPi129
atgggaatggcttctagagctgctagattggctgctagattggctaggctcgctctcagggcgctcgtcgacggtggaggcggatctggtggcggtggaagcgcggcccagccggccatggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 54 - DNA, 864 bp, Artificial
  coding sequence for Mag-(G4S)2-scFvPi129; fusion protein comprising Mag-(G4S)2-scFvPi129
atgggaggtattggtaagtttttgcattctgctggtaagtttggtaaggcttttgttggtgaaattatgaagtctgtcgacggtggaggcggatctggtggcggtggaagcgcggcccagccggccatggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 55 - DNA, 873 bp, Artificial
  coding sequence for MTK-(G4S)2-scFvPi129; fusion protein comprising MTK-(G4S)2-scFvPi129
atgggacatagaagagagggacctatttttgatactagaccttctccattcaatcctaatcaacctagacctggacctatttatgtcgacggtggaggcggatctggtggcggtggaagcgcggcccagccggccatggcacagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttacagatcaacaacctcaaaaatgaggatacggcaacttatttctgtgtaaggagggttgaggcttactggggccaagggactctggtcaccgtctcctcaggctccacctcaggctccggtaaacctggcccaggggagggatcaactaagggcgcgccacaaattgttctcacccagtctccagcaatcatgtctgcatctccaggggagaaggtcaccatgacctgcagtgccagctcaagtgtaagtcacatgcaatggtaccaacagaagtcaggcacctcccccaaaagatggatttatgacacatccaaactggcttctggagtccctgatcgtttcagtggcagtgggtctgggacctcttactctctcacaatcagcagcatggaggctgaagatggtgccacttattactgccagcagtggagtagtaacccatggacgttcggtggaggcaccaagctggaaatcaaacgggctgatgct

SEQ ID NO: 56 - DNA, 873 bp, Artificial
  coding sequence for Cec-(G4S)2-scFvPi68; fusion protein comprising Cec-(G4S)2-scFvPi68
atggctaagtggaagttgtttaagaagattttgaaggttttggtcgacggtggaggcggatctggtggcggtggaagcgcggcccagccggccatggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 57 - DNA, 888 bp, Artificial
  coding sequence for D4E1-(G4S)2-scFvPi68; fusion protein comprising D4E1-(G4S)2-scFvPi68
atgggatttaagttgagagctaagattaaggttagattgagagctaagattaagttggtcgacggtggaggcggatctggtggcggtggaagcgcggcccagccggccatggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 58 - DNA, 897 bp, Artificial
  coding sequence for GR7-(G4S)2-scFvPi68; fusion protein comprising GR7-(G4S)2-scFvPi68
atgggaatggcttctagagctgctagattggctgctagattggctaggctcgctctcagggcgctcgtcgacggtggaggcggatctggtggcggtggaagcgcggcccagccggccatggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 59 - DNA, 906 bp, Artificial
  coding sequence for Mag-(G4S)2-scFvPi68; fusion protein comprising Mag-(G4S)2-scFvPi68
atgggaggtattggtaagtttttgcattctgctggtaagtttggtaaggcttttgttggtgaaattatgaagtctgtcgacggtggaggcggatctggtggcggtggaagcgcggcccagccggccatggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 60 - DNA, 915 bp, Artificial
  coding sequence for MTK-(G4S)2-scFvPi68; fusion protein comprising MTK-(G4S)2-scFvPi68
atgggacatagaagagagggacctatttttgatactagaccttctccattcaatcctaatcaacctagacctggacctatttatgtcgacggtggaggcggatctggtggcggtggaagcgcggcccagccggccatggctgccgtgacgttggacgagtccgggggcggcctccagacgcccggaggagggctcagcctcgtctgcaaggcctccgggttcaccttcagcagcgtcagcatgggttgggtgcgacaggcgcccggcaaggggctggagttcgtcgcggctattagcagtggtagtggtagtaatatatactacgcgacagcggtgaagggccgtgccaccatctcgagggacaatgggcagagcacagtgaggctgcagctgagcaacctcagggctgaggacaccggcacctactactgcgccaaaaatgcttatggtactcctgattgttatattgctattggttgtactggtaacatcgacgcatggggccacgggaccgaggtcaccgtctcctcaggctccacctcaggctccggtaaacctggcccaggggagggatcaactaagggcgcgcctgcgctgactcagccgtcctcggtgtcagcaaacccaggagaaaccgtcaagatcacctgctccgggggtggtggcagctatggctggtatcagcagaagtctcctggcagtgcccctgtcactctgattcatggtagcagcagcagaccctcggacatcccttcacgattctccggttccaaatccggctccacagccacattaaccatcactggggtccaagccgaggacgaggctgtctacttctgtggtggctacgacagcagtaatcctggtatatttggggccgggacaaccctgaccgtcctaggccagcccgtcgac

SEQ ID NO: 61 - protein, 252 aa, Artificial
  scFvPi5 with specificity against Phytophthora infestans; originates from Mus musculus
MAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 62 - protein, 257 aa, Artificial
  scFvPi33 with specificity against Phytophthora infestans; originates from Mus musculus
MAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 63 - protein, 251 aa, Artificial
  scFvPi76.1 with specificity against Phytophthora infestans; originates from Mus musculus
MGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 64 - protein, 251 aa, Artificial
  scFvPi76.2 with specificity against Phytophthora infestans; originates from Mus musculus
MGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 65 - protein, 252 aa, Artificial
  scFvPi86 with specificity against Phytophthora infestans; originates from Mus musculus
MAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 66 - protein, 262 aa, Artificial
  scFvPi88 with specificity against Phytophthora infestans; originates from Mus musculus
MGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 67 - protein, 252 aa, Artificial
  scFvPi102.2 with specificity against Phytophthora infestans; originates from Mus musculus
MAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 68 - protein, 246 aa, Artificial
  scFvPi129 with specificity against Phytophthora infestans; originates from Mus musculus
MAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 69 - protein, 260 aa, Artificial
  scFvPi68 with specificity against Phytophthora infestans; originates from Gallus gallus
MAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD

SEQ ID NO: 70 - protein, 14 aa, Artificial
  Cec; originates from Hyalophora cecropia; synthetic Cecropin A-derived peptide
MAKWKLFKKILKVL

SEQ ID NO: 71 - protein, 19 aa, Artificial
  D4E1; synthetic peptide
MGFKLRAKIKVRLRAKIKL

SEQ ID NO: 72 - protein, 22 aa, Artificial
  GR7; synthetic peptide
MGMASRAARLAARLARLALRAL

SEQ ID NO: 73 - protein, 25 aa, Artificial
  Mag; originates from Xenopus laevis Magainin 1 peptide
MGGIGKFLHSAGKFGKAFVGEIMKS

SEQ ID NO: 74 - protein, 28 aa, Artificial
  MTK (Metchnikowin)
MGHRRQGPIFDTRPSPFNPNQPRPGPIY

SEQ ID NO: 75 - protein, 10 aa, Artificial
  (G4S)2 linker
GGGGSGGGGS

SEQ ID NO: 76 - protein, 283 aa, Artificial
  Cec-(G4S)2-scFvPi5; fusion protein comprising Cec-(G4S)2-scFvPi5
MAKWKLFKKILKVLVDGGGGSGGGGSAAQPAMAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 77 - protein, 288 aa, Artificial
  D4E1-(G4S)2-scFvPi5; fusion protein comprising D4E1-(G4S)2-scFvPi5
MGFKLRAKIKVRLRAKIKLVDGGGGSGGGGSAAQPAMAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 78 - protein, 291 aa, Artificial
  GR7-(G4S)2-scFvPi5; fusion protein comprising GR7-(G4S)2-scFvPi5
MGMASRAARLAARLARLALRALVDGGGGSGGGGSAAQPAMAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 79 - protein, 294 aa, Artificial
  Mag-(G4S)2-scFvPi5; fusion protein comprising Mag-(G4S)2-scFvPi5
MGGIGKFLHSAGKFGKAFVGEIMKSVDGGGGSGGGGSAAQPAMAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 80 - protein, 297 aa, Artificial
  MTK-(G4S)2-scFvPi5; fusion protein comprising MTK-(G4S)2-scFvPi5
MGHRREGPIFDTRPSPFNPNQPRPGPIYVDGGGGSGGGGSAAQPAMAEVQLQQSGPELVKPGASMKISCKASGYSFTGYTMNWMKQSHGKSLEWIGLINPYNGGSSYNQKFKGKATLTVDKSSSTAYMELLSLTSEDSAVYYCARGSTTGPYFDVWGAGTTVTVSSGSTSGSGKPGPGEGSTKGAPDIQMTQSTSSLSASLGDRVTINCRASQDISNYLNWYQQKPDGTVKLLIYYTSRLHSGVPSRFSGSGSGTDYSLTINNLEQEDIATYFCQQGHTLPYTFGGGTKLELKRAVD

SEQ ID NO: 81 - protein, 288 aa, Artificial
  Cec-(G4S)2-scFvPi33; fusion protein comprising Cec-(G4S)2-scFvPi33
MAKWKLFKKILKVLVDGGGGSGGGGSAAQPAMAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 82 - protein, 293 aa, Artificial
  D4E1-(G4S)2-scFvPi33; fusion protein comprising D4E1-(G4S)2-scFvPi33
MGFKLRAKIKVRLRAKIKLVDGGGGSGGGGSAAQPAMAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 83 - protein, 296 aa, Artificial
  GR7-(G4S)2-scFvPi33; fusion protein comprising GR7-(G4S)2-scFvPi33
MGMASRAARLAARLARLALRALVDGGGGSGGGGSAAQPAMAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 84 - protein, 299 aa, Artificial
  Mag-(G4S)2-scFvPi33; fusion protein comprising Mag-(G4S)2-scFvPi33
MGGIGKFLHSAGKFGKAFVGEIMKSVDGGGGSGGGGSAAQPAMAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 85 - protein, 302 aa, Artificial
  MTK-(G4S)2-scFvPi33; fusion protein comprising MTK-(G4S)2-scFvPi33
MGHRREGPIFDTRPSPFNPNQPRPGPIYVDGGGGSGGGGSAAQPAMAEVQLQQSGPELVKPGASVKMSCKTSGYTFTNYIMHWVKQQPGQGLEWIGYINPYNDATKYNEKFKGKATLTSDRSSSTAYMELSSLTSEDSAVYYCARVGDVSGFAYWGQGTLVTVSSGSTSGSGKPGPGEGSTKGAPDIVLSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLEIKRAVD

SEQ ID NO: 86 - protein, 282 aa, Artificial
  Cec-(G4S)2-scFvPi76.1; fusion protein comprising Cec-(G4S)2-scFvPi76.1
MAKWKLFKKILKVLVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 87 - protein, 287 aa, Artificial
  D4E1-(G4S)2-scFvPi76.1; fusion protein comprising D4E1-(G4S)2-scFvPi76.1
MGFKLRAKIKVRLRAKIKLVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 88 - protein, 290 aa, Artificial
  GR7-(G4S)2-scFvPi76.1; fusion protein comprising GR7-(G4S)2-scFvPi76.1
MGMASRAARLAARLARLALRALVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 89 - protein, 293 aa, Artificial
  Mag-(G4S)2-scFvPi76.1; fusion protein comprising Mag-(G4S)2-scFvPi76.1
MGGIGKFLHSAGKFGKAFVGEIMKSVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 90 - protein, 296 aa, Artificial
  MTK-(G4S)2-scFvPi76.1; fusion protein comprising MTK-(G4S)2-scFvPi76.1
MGHRREGPIFDTRPSPFNPNQPRPGPIYVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGETITLTCRSSTGAVTTSNYANWVQEKPDHLFTGLIGGTNNRAPGVPARFSGSLIGDKAALTITGAQTEDEAIYFCALWYSNHYVFGSGTKVTVLGQPK

SEQ ID NO: 91 - protein, 282 aa, Artificial
  Cec-(G4S)2-scFvPi76.2; fusion protein comprising Cec-(G4S)2-scFvPi76.2
MAKWKLFKKILKVLVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 92 - protein, 287 aa, Artificial
  D4E1-(G4S)2-scFvPi76.2; fusion protein comprising D4E1-(G4S)2-scFvPi76.2
MGFKLRAKIKVRLRAKIKLVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 93 - protein, 290 aa, Artificial
  GR7-(G4S)2-scFvPi76.2; fusion protein comprising GR7-(G4S)2-scFvPi76.2
MGMASRAARLAARLARLALRALVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 94 - protein, 293 aa, Artificial
  Mag-(G4S)2-scFvPi76.2; fusion protein comprising Mag-(G4S)2-scFvPi76.2
MGGIGKFLHSAGKFGKAFVGEIMKSVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 95 - protein, 296 aa, Artificial
  MTK-(G4S)2-scFvPi76.2; fusion protein comprising MTK-(G4S)2-scFvPi76.2
MGHRREGPIFDTRPSPFNPNQPRPGPIYVDGGGGSGGGGSAAQPAMGEVKLLESGGGLVQPGGSLKLSCAASGFDFSRYWMSWVRQAPGKGLEWIGEIKPDSSTINYTPSLKDKFIISRDNAKNSLYLQMSRVRSEDTALYYCARRGGYFAYWGQGTLVTVSSGSTSGSGKPGPGEGSTKGAPQAVVTQESALTTSPGGTVILTCRSSTGAVTTSNNANWVQEKPDHLFTGLIGGTSNRAPGVPVRFSGSLIGDKAALTITGAQTEDDAMYFCALWYSTHYVFGGGTKVTVLGQPK

SEQ ID NO: 96 - protein, 283 aa, Artificial
  Cec-(G4S)2-scFvPi86; fusion protein comprising Cec-(G4S)2-scFvPi86
MAKWKLFKKILKVLVDGGGGSGGGGSAAQPAMAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 97 - protein, 288 aa, Artificial
  D4E1-(G4S)2-scFvPi86; fusion protein comprising D4E1-(G4S)2-scFvPi86
MGFKLRAKIKVRLRAKIKLVDGGGGSGGGGSAAQPAMAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 98 - protein, 291 aa, Artificial
  GR7-(G4S)2-scFvPi86; fusion protein comprising GR7-(G4S)2-scFvPi86
MGMASRAARLAARLARLALRALVDGGGGSGGGGSAAQPAMAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 99 - protein, 294 aa, Artificial
  Mag-(G4S)2-scFvPi86; fusion protein comprising Mag-(G4S)2-scFvPi86
MGGIGKFLHSAGKFGKAFVGEIMKSVDGGGGSGGGGSAAQPAMAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 100 - protein, 297 aa, Artificial
  MTK-(G4S)2-scFvPi86; fusion protein comprising MTK-(G4S)2-scFvPi86
MGHRREGPIFDTRPSPFNPNQPRPGPIYVDGGGGSGGGGSAAQPAMAQVQLQQPGSVLVRPGASVKLSCKASGYTFTSSWMHWAKQRPGQGLEWIGEIHPNSGESHHNEKFKGKATLTVDTSTSTAYVDLSSLTSEDSAVYYCVRGQLGPSYWGQGTLVTVSSGSTSGSGKPGPGEGSTKGAPQAVVTQESALTTSPGETVTLTCRSSTGAVTTSNYANWVQEKPDHSFTSLIGGTNNRAPGVPARFSGSLIGDKAALTITGAQTEDEAIYFCALWYSNHFWVFGGGTKLTVLGQPK

SEQ ID NO: 101 - protein, 293 aa, Artificial
  Cec-(G4S)2-scFvPi88; fusion protein comprising Cec-(G4S)2-scFvPi88
MAKWKLFKKILKVLVDGGGGSGGGGSAAQPAMGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 102 - protein, 298 aa, Artificial
  D4E1-(G4S)2-scFvPi88; fusion protein comprising D4E1-(G4S)2-scFvPi88
MGFKLRAKIKVRLRAKIKLVDGGGGSGGGGSAAQPAMGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 103 - protein, 301 aa, Artificial
  GR7-(G4S)2-scFvPi88; fusion protein comprising GR7-(G4S)2-scFvPi88
MGMASRAARLAARLARLALRALVDGGGGSGGGGSAAQPAMGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 104 - protein, 304 aa, Artificial
  Mag-(G4S)2-scFvPi88; fusion protein comprising Mag-(G4S)2-scFvPi88
MGGIGKFLHSAGKFGKAFVGEIMKSVDGGGGSGGGGSAAQPAMGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 105 - protein, 307 aa, Artificial
  MTK-(G4S)2-scFvPi88; fusion protein comprising MTK-(G4S)2-scFvPi88
MGHRREGPIFDTRPSPFNPNQPRPGPIYVDGGGGSGGGGSAAQPAMGEVKLVESGGGLVQPGGSLRLSCATSGFTFSDFYMEWVRQPPGKRLEWIAASRNKANDYTTEYSASVKGRFFVSRDTSQSILYLQMNALRAEDTAIYYCARDYYGSSYWYFDVWGAGTTVTVSSGSTSGSGKPGPGEGSTKGAPDIVMTQSPTFLAVTASKKVTISCTASESLYSSKHKVHYLAWYQKKPEQSPKLLIYGASNRYIGVPDRFTGSGSGTDFTLTISSVQVEDLTHYYCAQFYRYPLTFGAGTKLELKRADA

SEQ ID NO: 106 - protein, 283 aa, Artificial
  Cec-(G4S)2-scFvPi102.2; fusion protein comprising Cec-(G4S)2-scFvPi102.2
MAKWKLFKKILKVLVDGGGGSGGGGSAAQPAMAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 107 - protein, 288 aa, Artificial
  D4E1-(G4S)2-scFvPi102.2; fusion protein comprising D4E1-(G4S)2-scFvPi102.2
MGFKLRAKIKVRLRAKIKLVDGGGGSGGGGSAAQPAMAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 108 - protein, 291 aa, Artificial
  GR7-(G4S)2-scFvPi102.2; fusion protein comprising GR7-(G4S)2-scFvPi102.2
MGMASRAARLAARLARLALRALVDGGGGSGGGGSAAQPAMAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 109 - protein, 294 aa, Artificial
  Mag-(G4S)2-scFvPi102.2; fusion protein comprising Mag-(G4S)2-scFvPi102.2
MGGIGKFLHSAGKFGKAFVGEIMKSVDGGGGSGGGGSAAQPAMAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 110 - protein, 297 aa, Artificial
  MTK-(G4S)2-scFvPi102.2; fusion protein comprising MTK-(G4S)2-scFvPi102.2
MGHRREGPIFDTRPSPFNPNQPRPGPIYVDGGGGSGGGGSAAQPAMAQVQLQQPGFVLVRPGASVKLSCKASGYSFTSTWMHWAKQRPGQGLEWIGEIHPNSGDTHYNEKFKGKATLTVDTSSSTAYVDLSSLTSEDSAVYYCVRGPLGPSYWGQGTLVTVSSGSTSGSGKPGPGEGSTKGAPQAVVTQESALTTSPGETVTLTCRSSTGAVTTSNYANWVQEKPDHLFTGLISGTNNRAPGVPARFSGSLIGDKAALTITGAQTEDEAIYFCALWYSNHFWVFGGGTKLTVLGQPK

SEQ ID NO: 111 - protein, 277 aa, Artificial
  Cec-(G4S)2-scFvPi129; fusion protein comprising Cec-(G4S)2-scFvPi129
MAKWKLFKKILKVLVDGGGGSGGGGSAAQPAMAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 112 - protein, 282 aa, Artificial
  D4E1-(G4S)2-scFvPi129; fusion protein comprising D4E1-(G4S)2-scFvPi129
MGFKLRAKIKVRLRAKIKLVDGGGGSGGGGSAAQPAMAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 113 - protein, 285 aa, Artificial
  GR7-(G4S)2-scFvPi129; fusion protein comprising GR7-(G4S)2-scFvPi129
MGMASRAARLAARLARLALRALVDGGGGSGGGGSAAQPAMAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 114 - protein, 288 aa, Artificial
  Mag-(G4S)2-scFvPi129; fusion protein comprising Mag-(G4S)2-scFvPi129
MGGIGKFLHSAGKFGKAFVGEIMKSVDGGGGSGGGGSAAQPAMAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 115 - protein, 291 aa, Artificial
  MTK-(G4S)2-scFvPi129; fusion protein comprising MTK-(G4S)2-scFvPi129
MGHRREGPIFDTRPSPFNPNQPRPGPIYVDGGGGSGGGGSAAQPAMAQIQLVQSGPELKKPGETVKISCKASGYTFTNYGMNWVKQAPGKGLKWMGWINTYTGEPTYVDDFKGRFAFSLETSASTAYLQINNLKNEDTATYFCVRRVEAYWGQGTLVTVSSGSTSGSGKPGPGEGSTKGAPQIVLTQSPAIMSASPGEKVTMTCSASSSVSHMQWYQQKSGTSPKRWIYDTSKLASGVPDRFSGSGSGTSYSLTISSMEAEDGATYYCQQWSSNPWTFGGGTKLEIKRADA

SEQ ID NO: 116 - protein, 291 aa, Artificial
  Cec-(G4S)2-scFvPi68; fusion protein comprising Cec-(G4S)2-scFvPi68
MAKWKLFKKILKVLVDGGGGSGGGGSAAQPAMAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD

SEQ ID NO: 117 - protein, 296 aa, Artificial
  D4E1-(G4S)2-scFvPi68; fusion protein comprising D4E1-(G4S)2-scFvPi68
MGFKLRAKIKVRLRAKIKLVDGGGGSGGGGSAAQPAMAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD

SEQ ID NO: 118 - protein, 299 aa, Artificial
  GR7-(G4S)2-scFvPi68; fusion protein comprising GR7-(G4S)2-scFvPi68
MGMASRAARLAARLARLALRALVDGGGGSGGGGSAAQPAMAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD

SEQ ID NO: 119 - protein, 302 aa, Artificial
  Mag-(G4S)2-scFvPi68; fusion protein comprising Mag-(G4S)2-scFvPi68
MGGIGKFLHSAGKFGKAFVGEIMKSVDGGGGSGGGGSAAQPAMAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD

SEQ ID NO: 120 - protein, 305 aa, Artificial
  MTK-(G4S)2-scFvPi68; fusion protein comprising MTK-(G4S)2-scFvPi68
MGHRREGPIFDTRPSPFNPNQPRPGPIYVDGGGGSGGGGSAAQPAMAAVTLDESGGGLQTPGGGLSLVCKASGFTFSSVSMGWVRQAPGKGLEFVAAISSGSGSNIYYATAVKGRATISRDNGQSTVRLQLSNLRAEDTGTYYCAKNAYGTPDCYIAIGCTGNIDAWGHGTEVTVSSGSTSGSGKPGPGEGSTKGAPALTQPSSVSANPGETVKITCSGGGGSYGWYQQKSPGSAPVTLIHGSSSRPSDIPSRFSGSKSGSTATLTITGVQAEDEAVYFCGGYDSSNPGIFGAGTTLTVLGQPVD